(19) United States
(12) Patent Application Publication
(10) Pub. No.: US 2022/0265892 A1
(43) Pub. Date: Aug. 25, 2022

(54) ABSORBENT ARTICLE WITH A HYDROPHILIC HOT MELT ADHESIVE

(71) Applicant: The Procter & Gamble Company, Cincinnati, OH (US)

(72) Inventors: Torsten LINDNER, Kronberg (DE); Gültekin ERDEM, Beijing (CN); Jinyu CHEN, Fanwood, NJ (US); Yuhong HU, Belle Mead, NJ (US)

(21) Appl. No.: 17/676,915

(22) Filed: Feb. 22, 2022

(30) Foreign Application Priority Data
Feb. 22, 2021 (CN) PCT/CN2021/077211

(51) Int. Cl. A61L 15/24; A61L 15/58

(52) U.S. Cl. CPC A61L 15/24; A61L 15/58

(57) ABSTRACT

Absorbent articles, such as diapers or sanitary napkins, comprising a hydrophilic hot melt adhesive with water wetting properties. The hydrophilic hot melt adhesive comprises an ethylene copolymer selected from the group consisting of ethylene vinyl acetate, ethylene n-butyl acrylate, ethylene octene, ethylene propylene, ethylene butene propylene, and mixtures thereof and a nonionic, fatty alcohol ethoxylate surfactant. The hot melt adhesive is particularly suitable for bonding a nonwoven to another component of the article.

### CROSS REFERENCE TO RELATED APPLICATION

This application claims priority, under 35 U.S.C. 119(b), to PCT Patent Application No. PCT/CN2021/077211, filed on Feb. 22, 2021, which is incorporated herein by reference.

### FIELD

The present disclosure relates to personal hygiene absorbent articles, such as diapers or feminine protections, comprising a hydrophilic hot melt adhesive. The hydrophilic adhesive can help a fluid such a urine to transfer from one layer of the article to another. The hydrophilic hot melt adhesive may in particular be used to bond a nonwoven to another layer, which may be a second nonwoven or an absorbent material.

### BACKGROUND

Nonwovens are used commercially in disposable articles such as diapers, adult incontinent products and sanitary napkins, in particular as topsheet, acquisition layer or core wrap for the absorbent core. In these applications, it is necessary to adhere the nonwoven to another substrate or component. The second substrate may be another nonwoven, tissue, or an unrelated material such as an absorbent material. A commonly employed technique to bond the assembly together is the use of a hot melt adhesive. The hot melt adhesives allow for cost and time efficient manufacturing since there is no evaporation step necessary as is the case for water based or solvent based adhesive systems. Suitable hot melt adhesives must possess excellent adhesion to the substrates involved. For nonwoven applications they must also possess good flexibility, no staining or bleed through, suitable viscosity, set speed and open time to function on commercially available equipment, and acceptable thermal aging properties.

In personal hygiene article such as disposable diapers, sanitary napkins and bed pad constructions, it is desired to draw the moisture away from the body and into the absorbent core as quickly as possible after the article is wetted. A hot melt adhesive having the ability to facilitate the transmission of the liquid from the nonwoven substrate towards the absorbent material is thus desirable.

WO 00/00229 (Lindner et al.) discloses hygienic articles comprising an oil resistant, hydrophilic adhesive. The formula of the adhesive or its permanency is however not disclosed. U.S. Pat. No. 6,380,292B1 (Bostik) discloses hydrophilic hot melt adhesive compositions suitable for nonwoven disposable articles which are prepared by blending various adhesive components with a surfactant.

There is a need for further hydrophilic adhesive compositions that are safe for use in personal absorbent articles, retain their hydrophilic properties over time and are cost-effective in-use and in production.

### BRIEF SUMMARY

The present disclosure is directed to absorbent articles, which are typically disposable such as diapers or female sanitary napkins, comprising a hydrophilic hot melt adhesive composition with improved water wetting properties. The hydrophilic hot melt adhesive composition comprises a) an ethylene copolymer selected from the group consisting of ethylene vinyl acetate, ethylene n-butyl acrylate, ethylene octene, ethylene propylene, ethylene butene propylene and mixtures thereof; b) at least one nonionic, fatty alcohol ethoxylate surfactant having (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E-222, and (iv) a molecular weight of about 400 to about 5000 Daltons.

In a second aspect, the hydrophilic hot melt adhesive comprises:
(a) from about 20 to about 90 wt% of an ethylene copolymer selected from the group consisting of ethylene vinyl acetate, ethylene n-butyl acrylate, ethylene octene, ethylene propylene, ethylene butene propylene and mixtures thereof; and
(b) from about 1 to about 25 wt% of one or more nonionic, fatty alcohol ethoxylate surfactant having (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E222, and (iv) a molecular weight of about 400 to about 5000 Daltons.

Other aspects of the invention are described herein and in the appended claims.

The hydrophilic hot melt adhesive may be used to attach a first nonwoven to another component of the absorbent article, such as a second nonwoven, fibers such as cellulose fibers, or superabsorbent particles. The hydrophilic hot melt adhesive advantageously exhibits a film contact angle with distilled water of less than 70 degrees, preferably less than 50 degrees, even after ageing at 15 days at 60° C.

### DETAILED DESCRIPTION

### Preamble

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values. As used herein, approximating language may be applied to modify any quantitative representation that may vary without resulting in a change in the basic function to which it is related. Accordingly, a value modified by a term or terms, such as “about,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11 ”, and “about 1” may mean from 0.9-1.1. Other meanings of “about” may be apparent from the context, such as rounding off, so, for example “about 1” may also mean from 0.5 to 1.4.

All % are by weight of the adhesive composition unless indicated otherwise.

### Absorbent Article

“Absorbent articles”, as used herein, refers to personal hygiene products that are placed against or in proximity to the body of the wearer to absorb and contain the various exudates discharged from the body. Absorbent articles include baby diapers, training pants, adult incontinence undergarments, feminine hygiene products, bed matt, changing matt and the like. In another form, the absorbent article may be an insert for use with a reusable outer cover. As used herein, the term “body fluids” or “body exudates” includes, but is not limited to, urine, blood, vaginal discharges and fecal matter. These articles are typically disposable, meaning are not intended to be laundered or otherwise restored or reused as a hygienic article after a single use. Disposable absorbent articles typically comprise a liquid pervious topsheet, a liquid impervious backsheet and an absorbent core positioned between the topsheet and the backsheet.

The topsheet is liquid pervious, permitting liquids (e.g., menses and/or urine) to readily penetrate through its thickness. A suitable topsheet may be manufactured from a wide range of materials such as woven and nonwoven materials (e.g., a nonwoven web of fibers); polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. When the topsheet comprises a nonwoven web, the web may be manufactured by a wide number of known techniques. For example, the web may be spunbonded, carded, wet-laid, melt-blown, hydroentangled, combinations of the above, or the like.

The backsheet is impervious to liquids (e.g., menses and/or urine) and is preferably manufactured from a thin plastic film, although other flexible liquid impervious materials may also be used. The backsheet prevents the exudates absorbed and contained in the absorbent core from wetting articles which contact the absorbent article such as bedsheets, pants, pajamas and undergarments. The backsheet may thus comprise a woven or nonwoven material, polymeric films such as thermoplastic films of polyethylene or polypropylene, or composite materials such as a film-coated nonwoven material. A suitable backsheet is a polyethylene film having a thickness of from about 0.012 mm (0.5 mil) to about 0.051 mm (2.0 mils). The backsheet is preferably embossed and/or matte finished to provide a more clothlike appearance. Further, the backsheet may permit vapors to escape from the absorbent core (i.e., the backsheet is breathable) while still preventing exudates from passing through the backsheet. The size of the backsheet is dictated by the size of the absorbent core and the exact absorbent article design selected.

Personal hygiene absorbent articles typically comprise an absorbent structure disposed between topsheet and backsheet for absorbing and containing liquid such as urine received by the absorbent article, referred to as absorbent core. The absorbent core is the component of the absorbent article having the most absorbent capacity. The absorbent core comprises an absorbent material, that may be disposed within a core wrap if the absorbent material does not have sufficient integrity on its own. The core wrap may be a single material that is folded and attached to itself, or it may comprise a separate top layer and bottom layer that are bonded together. The absorbent material typically comprises superabsorbent particles which are optionally mixed with cellulose fibers. As used herein, “absorbent core” does not include any acquisition layer, distribution layer, acquisition-distribution system, topsheet, or backsheet of the absorbent article.

The absorbent core may typically have a generally rectangular shape as seen from above when the article is laid flat on a surface. The absorbent core comprises an absorbent material layer which may have a generally rectangular outline, or a non-rectangular outline (“shaped” core), in particular the absorbent material layer may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “sand-hour” for the area of the absorbent material.

The absorbent material may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores where the absorbent material consists of SAP.

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

Various absorbent core designs comprising high amount of SAP have been proposed in the past, see for example in US 5,599,335 (Goldman), EP 1,447,066 (Busam), WO 95/11652 (Tanzer), US 2008/0312622 A1 (Hundorf), WO 2012/052172 (Van Malderen). In particular the SAP printing technology as disclosed in US 2006/0024433 (Blessing), US 2008/0312617 and US 2010/0051166 A1 (both to Hundorf et al.) may be used. The present disclosure is however not limited to a particular type of absorbent core. The absorbent core may also comprise one or more glue such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity.

The absorbent material may be deposited as a continuous layer within the core wrap. The absorbent material may also be present discontinuously for example as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern wherein the resulting layer is substantially continuously distributed across the absorbent particulate polymer material area. As for example taught in US 2008/0312622 A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise longitudinally extending channels which are substantially free of absorbent material within the absorbent material area. The core wrap may be bonded through these material-free areas. Exemplary disclosures of such channels in an airfelt-free core can be found in WO 2012/170778 (Rosati et al.) and US 2012/0312491 (Jackels). Channels may of course also be formed in absorbent cores comprising cellulose fibers.

The absorbent core is disposed between the backsheet and the topsheet, including the possible presence of other optional layers such as an acquisition layer. The function of the acquisition layer is to quickly draw the fluid away from the topsheet to keep the skin as dry.

Acquisition layers are typically disposed between the topsheet and the absorbent core. Typical examples of acquisition layers are air-through bonded carded web, with a basis weight ranging typically from 10 gsm to 60 gsm, as is known in the art. Some absorbent articles further have a distribution layer in addition to an acquisition layer, whose function is to distribute the fluid from a region of insult to a larger surface. A typical material for a distribution layer is a layer of loosely bonded cross-linked cellulose fibers as described in the references above (e.g., US 2008/0312622 A1), in order to maximize the speed of absorption of the absorbent core.

### Hot Melt Adhesive

Conventional hot melt adhesives are hydrophobic in nature and form a barrier to the transfer of fluid between layers, in particular between the topsheet and the absorbent core, hindering aqueous materials, e.g., moisture, water, blood, urine, menses, to flow into the core. The present hydrophilic hot melt adhesive on the other hand facilitates the transmission of the aqueous materials from the topsheet to the superabsorbent core or fluff core substrate due to its low contact angle. The hydrophilic hot melt adhesive may be used as a construction adhesive between different layers or components of the article, e.g., between the topsheet and acquisition layer, or acquisition layer and absorbent core, or between absorbent material and top core wrap layer. The hotmelt adhesives of the present disclosure are also compatible with typical skincare lotions that may be present on the topsheet or used by a caregiver.

The hydrophilic hot melt adhesive of the present disclosure comprises an ethylene copolymer selected from an ethylene vinyl acetate, ethylene n-butyl acrylate, ethylene octene, ethylene propylene, ethylene butene propylene and mixtures thereof. The ethylene monomer may have a content as low as 5 wt % of the copolymer; however, ethylene homopolymers are not a preferred polymer for the hydrophilic hot melt adhesive.

The total amount of ethylene copolymer is typically present in the hydrophilic adhesive composition in amounts ranging from about 20% to about 90%, by weight of the hot melt adhesive composition, and preferably from about 30% to about 70% by weight of the hot melt adhesive composition. The ethylene copolymer may be provided as a single ethylene copolymer material, but it may be also provided by a mixtures of ethylene copolymer materials as defined above, if desired.

The hydrophilic hot melt adhesive further comprises a nonionic, ethoxylate surfactant. The nonionic, ethoxylate surfactant may comprise a single nonionic, ethoxylate surfactant material, or if desired a mixture thereof. The nonionic, ethoxylate surfactants used herein have a relatively high molecular weight ranging from about 400 to about 5000 Daltons, preferably from about 500 to about 3000 Daltons. The melting point of the ethoxylated surfactants range from 70° C. to about 120° C., preferably from about 80 to about 100° C., more preferably from about 85 to about 95° C.

The ethoxylated surfactant is derived from a fully saturated, long linear chain C20 to C50 synthetic alcohols. This long polymer chain allows for easy incorporation into the polymer system of the adhesive, and its thermostability is significantly improved. The polymer surfactant provides bulk strength and allows for good wetting properties. The ethoxylated surfactant may have a hydrophile-lipophile balance (HLB) number of less than 20, and is incorporated such that a film formed by the resultant adhesive film preferably has a contact angle of 70° or less, and more preferably less than about 50°, in Fresh and Aged conditions as discussed further below.

The nonionic, ethoxylate surfactant differs from conventional nonionic ethoxylate alcohols and phenols. The conventional nonionic ethoxylate alcohols and phenols are converted into R(OC2H4)nOH, where n ranges from 1 to 10; and they are low molecular weight emulsifiers, have short chains, and have much lower melting temperatures, typically molten at ambient temperature. These conventional, low molecular weight nonionic materials include ATMER 688 from Croda, and these described in WO 97/48779 and U.S. Pat. No. 6,380,292 fail to provide low contact angles. Therefore, conventional nonionic ethoxylate alcohols and phenols are very mobile and cannot be stabilized in a polymer matrix. As a result, they will easily migrate to the surface of a polymer matrix and phase separate from the matrix, even at room temperature. Furthermore, given their mobility in the system, their capability to decrease the contact angle is unstable and short-lived. On the contrary, the addition of a nonionic, ethoxylate surfactant that have high molecular weight, long chain and high melting temperature, provides stability in the polymer matrix, and as a result, the contact angle reduction is consistent and durable, as evidenced in the unchanged contact angle after accelerated ageing tests.

The overall amount of nonionic, ethoxylate surfactant(s) in the hydrophilic adhesive composition may typically range of from about 1% to about 25%, by weight, and preferably from about 1% to about 10%. In case more than one of such surfactant is formulated, the weight amount of each is added for the overall weight % of the surfactant component.

The ethoxylated surfactant must be reasonably compatible with the other raw materials used in the hot melt adhesive so that it does not adversely affect the construction performance of the adhesive while at the same time the ethoxylated surfactant should be able to “bloom” to the surface of the adhesive so as to lower the contact angle and make the adhesive more hydrophilic. Thus, a delicate balance of compatibility must be maintained.

Optional components may be further added to the hydrophilic adhesive, such as tackifier, plasticizer, antioxidant, and the like.

The tackifying resins useful in the adhesive compositions can be hydrocarbon resins, synthetic polyterpenes, rosin esters, natural terpenes, and the like. More particularly, and depending upon the particular base polymer, the useful tackifying resins may include any compatible resins or mixtures thereof such as (1) natural and modified rosins such, for example, as gum rosin, wood rosin, talloil rosin, distilled rosin, hydrogenated rosin, dimerized rosin, and polymerized rosin; (2) glycerol and pentaerythritol esters of natural and modified rosins, such, for example as the glycerol ester of pale, wood rosin, the glycerol ester of hydrogenated rosin, the glycerol ester of polymerized rosin, the pentaerythritol ester of hydrogenated rosin, and the phenolic-modified pentaerythritol ester of rosin; (3) copolymers and terpolymers of natured terpenes, e.g., styrene/terpene and alpha methyl styrene/terpene; (4) polyterpene resins having a softening point, as determined by ASTM method E28-58T, of from about 80° to 150° C.; the latter polyterpene resins generally resulting from the polymerization of terpene hydrocarbons, such as the bicyclic monoterpene known as pinene, in the presence of Friedel-Crafts catalysts at moderately low temperatures; also included are the hydrogenated polyterpene resins; (5) phenolic modified terpene resins and hydrogenated derivatives thereof such, for example, as the resin product resulting from the condensation, in an acidic medium, of a bicyclic terpene and a phenol; (6) aliphatic petroleum hydrocarbon resins having a Ball and Ring softening point of from about 70° to 135° C.; the latter resins resulting from the polymerization of monomers consisting of primarily of olefins and diolefins; also included are the hydrogenated aliphatic petroleum hydrocarbon resins; (7) aromatic petroleum hydrocarbon resins and the hydrogenated derivatives thereof; and (8) alicyclic petroleum hydrocarbon resins and the hydrogenated derivatives thereof. Mixtures of two or more of the above described tackifying resins may be required for some formulations.

While the tackifier may comprise up to about 70% of the adhesive, it is generally used in amounts of about 20 to 50% by weight of the adhesive composition.

A plasticizer can be present in the composition of the present disclosure in amounts of up to about 20% by weight, preferably up to about 10 wt%, based on the total weight of the adhesive, in order to provide desired viscosity control without substantially decreasing the adhesive strength, the service temperature of the adhesive, and hydrophilicity of the adhesive. A suitable plasticizer may be selected from the group which not only includes the usual plasticizing oils, such as mineral oil, naphthenic, paraffinic, Gas to Liquid (GTL) oil, but also olefin oligomers and low molecular weight polymers, as well as vegetable and animal oil and derivatives of such oils. The petroleum derived oils which may be employed are relatively high boiling temperature materials containing only a minor proportion of aromatic hydrocarbons. In this regard, the aromatic hydrocarbons should preferably be less than 30%, and more particularly less than 15%, by weight, of the oil.

Alternately, the oil may be totally non-aromatic. The oligomers may be polypropylenes, polybutenes, hydrogenated polyisoprene, hydrogenated butadiene, or the like having average molecular weights between about 350 and about 10.000. Suitable vegetable and animals oils include glycerol esters of the usual fatty acids and polymerization products thereof. The plasticizer that finds usefulness in the present disclosure can be any number of different plasticizers but the inventors have discovered that mineral oil such as Kaydol manufactured by Witco, is particularly useful. Benzoflex 9-88, a dipropylene glycol dibenzoate manufactured by Velsicol, has also been found to be an appropriate plasticizer. Risalla X 409, a Gas to Liquid oil from Shell, is also suitable for a plasticizer. As will be appreciated, plasticizers have typically been employed to lower the viscosity of the overall adhesive composition without substantially decreasing the adhesive strength and/or the service temperature of the adhesive. The choice of plasticizer can be useful in formulation for specific end uses (such as wet strength core applications).

Among the applicable stabilizers or antioxidants which may be included herein are high molecular weight hindered phenols and multifunctional phenols such as sulfur and phosphorous-containing phenols. Representative hindered phenols include: 1,3,5-trimethyl-2,4,6-tris(3,5-di-tert-butyl-4-hydroxyphenyl) propionate; n-octadecyl 3,5-di-tert-butyl-4-hydroxyphenyl) propionate; 4,4′-methlenebis (2,6-di-tert-butylphenol); 4,4′-thiobis (6-tert-butyl-o-cresol); 2,6-di-tert-butylphenol; 6-(4-hydroxyphenoxy)-2,4-bis(n-octylthio)-1,3,5-triazine; di-n-octadecyl-3,5-di-tert-butyl-4-hydroxy-benzylphosphonate; 2-(n-octylthio)-ethyl 3 ,5-di-tert-butyl-4-hydroxybenzoate; and sorbitol hexa[3-(3,5-di-tert-butyl-4-hydroxyphenyl)propionate]. If used, the stabilizer is present in levels of about 0.1 to 3% by weight.

Optional additives may be incorporated into the hot melt compositions in order to modify certain properties thereof. Among these additives may be included wax, colorants such as titanium dioxide; and fillers such as talc and clay, etc.

A low contact angle is desirable so that water, urine or other water-based discharges “wet out” rather than “bead up” resulting in the fluid being directed into the core and away from the topsheet. The hydrophilicity of an adhesive can be quantified by the Adhesive Contact Angle Measurement described hereinafter. In order to be particularly suitable for the absorbent article of the present disclosure, the adhesive preferably have a contact angle with distilled water of less than 70°, more preferably less than 50° C.

The hydrophilic hot melt adhesives of the disclosure may be formulated using techniques known in the art. An exemplary procedure involves placing all the polymer, surfactant, tackifiers, plasticizers and stabilizers in a jacketed mixing kettle, preferably in a jacketed heavy duty mixer, which is equipped with rotors and thereupon raising the temperature to a range of from about 120° C. up to about 190° C. After the resin has melted, the temperature is lowered to a temperature of from 100° C. to 165° C. Mixing and heating are continued until a smooth, homogeneous mass is obtained. The solidified hydrophilic hot melt adhesive composition can then be pelletized or formed into blocks.

### Application

The hydrophilic hot melt adhesive may be applied onto various substrates including film, nonwoven, cellulose or synthetic fibers, or superabsorbent particles. The hydrophilic hot melt adhesive may be in particular used to bond a first component which is a nonwoven to a second component of the absorbent articles, which may be a second nonwoven, or cellulose fibers, or superabsorbent particles, or a combination thereof. The hydrophilic hot melt adhesive facilitates the transfer of liquid such as urine from one component to the next component thanks to its hydrophilicity through the use of the article, which may be several hours and cover several liquid insults. The hydrophilic hot melt adhesive may be used for example to attach the topsheet to an acquisition layer, an acquisition layer to a distribution layer, an acquisition or a distribution layer to the absorbent core, a topsheet directly or indirectly to the absorbent core. The core wrap, in particular the inner surface of the top side of the core wrap, may also be attached to the absorbent layer by the hot melt adhesive of the present disclosure to facilitate the transfer of liquid from the surface of the core wrap into the absorbent layer. The absorbent layer may be a mixture of cellulose fibers and superabsorbent particles, or superabsorbent particles substantially free of cellulose fibers. The absorbent layer, in particular for absorbent layer consisting of superabsorbent particles without cellulose fibers, may also be immobilized within the core wrap by a hot melt adhesive according to the present disclosure.

The adhesive may be applied using multi-line, spray, or slot-coating construction techniques. The adhesive may be in patterned layer of adhesive, or an array of separate lines, spirals, or spots of adhesive. An exemplary attachment means of an open pattern network of filaments comprises several lines of adhesive filaments swirled into a spiral patter. At least one liquid pervious substrate may be bonded to at least one tissue, non-woven, polyolefin or other flexible polymeric film substrate. In addition, the adhesive of the present disclosure can also be used as a hydrophilic coating to reduce contact angle of the substrates and to enhance hydrophilicity of the substrate surfaces. This can help driving the fluid transferring from a first component to a second component, for example from the topsheet to the acquisition layer, and/or the acquisition/distribution layer to the absorbent core, and/or from the core wrap to the absorbent material within the absorbent core.

### Test Methods

Viscosity: Viscosity was measured with a Brookfield viscometer, spindle #27 at 150° C., in accordance with ASTM 3236-88.

Contact Angle Test: As a drop of liquid meets a solid surface, it assumes a distinctive shape. The shape and length of time that it holds onto its shape are determined by three interfacial tension forces: the force of the solid surface, the surface tension of the liquid and the force at the solid/liquid interface. The contact angle (0) is a measured value relative to the combined vector forces according to the formula:

γ_{L} COSθ=γ_{S}−γ_{SL}

where γ_{L} is the interfacial tension of the liquid/air boundary, γ_{S} is the interfacial tension of the solid/air boundary, γ_{SL} is the interfacial tension of the solid/liquid boundary, and θ is the angle of the liquid drop.

The goniometer has a microsyringe for dispersing accurate droplet sizes and a camera for photographing the angle of the liquid drop as it meets the surface of the, solid. The contact angle is measured as the angle between the substrate and the tangent of the liquid drop (at the interface).

The lower the angle, the more effective the coating is in transmitting the liquid through the adhesive layer. The contact angle for Fresh and Aged film sample is measured as follows:

Fresh sample: a 50 micrometer thick film of adhesive coating is made on a PET film by a hot melt coater laminator (e.g. HLCL-1000, ChemInstruments) at 120° C. (adapt temperature if needed) and then let it cool down to ambient lab conditions (21-23° C., 40%-60% RH). The contact angle testing is conducted on the adhesive surface 24 hour after the adhesive coating making.

Aged sample: the fresh sample is put into a convection oven (e.g., Blue M convection oven, Stabil-Therm) and subjected to 15 days of aging. The oven temperature is set to 60° C. After ageing, the sample is taken out from the oven and conditioned at ambient lab conditions (21-23° C., 40%-60% RH) for 24 hours before the contact angle on adhesive surface is measured.

Instron Testing: The bond strength was evaluated on Instron Tester. The laminate bonded with hot melt adhesive was separated at the ends and placed in tensile tester jaws. The sample was then pulled at 12 in/min crosshead speed, and the average peel value recorded in grams or pounds for each product tested. If there was bond failure, the type of failure was recorded instead of peel value.

### Materials Used in the Examples

Table 1 lists the ethoxylate surfactant and their properties used in the examples.

TABLE 1

| | | Ethylene | | Hydroxyl | |
|---|---|---|---|---|---|
| | | oxide (% | | number (mg | |
| | Calculated | by weight) - | | KOH/g | Melting point |
| Ethoxylated | Mw | NMR | | sample) - | (° C.)-ASTM |
| surfactant | (Daltons) | calculation | HLB value | ASTM E-222 | D-127 |
| Unithox 420* | 575 | 20 | 4 | 85 | 91 |
| Unithos 450* | 920 | 50 | 10 | 55 | 91 |
| Unithox 480* | 2300 | 80 | 16 | 22 | 86 |

*Baker Hughes

Table 2 lists all the polymers used in the examples and their properties.

TABLE 2

| | | | | Ring& Ball | |
|---|---|---|---|---|---|
| | Polymer | Ethylene | Melt Flow | Softening | |
| Polymer Name | Type | content (%) | (g/10 min)* | Point (° C.)** | Tm (° C.) *** |
| Ateva¹ 3342 | EVA | 67 | 400 | 81 | |
| Ateva¹ 4030 | EVA | 60 | 55 | 107 | 52 |
| Ateva¹ 2842 | EVA | 72 | 400 | 84 | 65 |
| Ateva¹ 1850 | EVA | 82 | 150 | 95 | 84 |
| Enable² 33331 | EnBA | 67.5 | 330 | | 62 |
| Affinity³1875 | PE-Octene | 65 | 1250 | | 70 |
| Vistamaxx²8780 | PP-E | 12 | Viscosity | | 96 |
| | | | 3980 cps at | | |
| | | | 190° C. | | |
| Vestplast⁵ 508 | PP/B/E | 3 | Viscosity | 84 | |
| | | | 8000 cps at | | |
| | | | 190° C. | | |
| RT 2830⁶ | PP-B | 0 | Viscosity | 90 | |
| | | | 3000 cps at | | |
| | | | 190° C. | | |

*ASTM D 1238 @ load 2.16 kg @ 190° C.
**ASTM E 28
*** ASTM D 3418
¹Celanese
²ExxonMobile
³Dow
⁴ Kraton
⁵Evonik
⁶Rextac

### Formulation Examples

Table 3 demonstrates the contact angle of each formulation with various polymers. The representative tackifier used in the example is Escorez 5690, an aromatic modified cycloaliphatic hydrocarbon resins with softening point of 90.5° C. from ExxonMobil. The representative plasticizer used in the examples is Calsol 5550, a naphthenic oil from Calumet. The representative antioxidant used in the examples is Evernox 10 from Everspring.

Film contact angles measured 24 hours after making the samples (“Fresh” samples) and aged for 15 days at 60° C. (“Aged” samples) are shown in Table 3. Film contact angle with distilled water of less than 70 degrees both before and after aging are considered as pass (P). Contact angles greater than 70 degrees is considered as fail (F), as described in the comments.

TABLE 3

| Example | 1 | 2 | 3 | 4 | 5 | 6 | 7 | 8 | 9 | 10 |
|---|---|---|---|---|---|---|---|---|---|---|
| Escorez | | | | 45 | 45 | 45 | 45 | 45 | 45 | 45 |
| 5690 | | | | | | | | | | |
| Unithox | | 5 | 10 | | 18 | 18 | 18 | 18 | 18 | 18 |
| 450 | | | | | | | | | | |
| Ateva | 100 | 95 | 90 | 54 | 36 | | | | | |
| 2842 | | | | | | | | | | |
| Enable | | | | | | 36 | | | | |
| 33331 | | | | | | | | | | |
| Vistamax | | | | | | | 36 | | | |
| x 8780 | | | | | | | | | | |
| RT 2830 | | | | | | | | 36 | | |
| Vestoplast | | | | | | | | | 36 | |
| 508 | | | | | | | | | | |
| Affinity | | | | | | | | | | 36 |
| 1875 | | | | | | | | | | |
| Evernox | | 1 | 1 | 1 | 1 | 1 | 1 | 1 | 1 | 1 |
| 10 | | | | | | | | | | |
| Viscosity | | 44150 | 30000 | 10150 | 2000 | 2375 | 470 | 1220 | 1950 | 575 |
| @ | | | | | | | | | | |
| 150° C. | | | | | | | | | | |
| (cps) | | | | | | | | | | |
| Contact | 82 | 37 | 39 | 91 | 31 | 30 | 30 | 74 | 41 | 23 |
| Angle- | | | | | | | | | | |
| Fresh (°) | | | | | | | | | | |
| Contact | 87 | 34 | 38 | 91 | 33 | 27 | 40 | 87 | 35 | 30 |
| Angle-15 | | | | | | | | | | |
| days at | | | | | | | | | | |
| 60° C. (°) | | | | | | | | | | |
| Comment | F | P, but | P | F | P | P | P | F | P | P |
| | | high | | | | | | | | |
| | | vis- | | | | | | | | |
| | | cosity | | | | | | | | |

Pure polymers, represented by example 1 (Ateva 2842 alone) have relatively high contact angle, greater than 80° in this example 1 has a contact angle of 82° and 87° after aging at 60° C. for 15 days. Without the nonionic ethoxylate surfactant, the contact angle remains high, Example 4. When this polymer is mixed with ethoxylate surfactant, Example 2, its thin film contact angle is substantially reduced to 37° for fresh sample, and 34° after aging at high temperature.

The ethylene-based polymer has a good compatibility with the nonionic ethoxylate surfactant, leading to a homogeneous system that is critical for the stable wetting properties of end adhesive. As seen, both fresh and aged thin film maintain low contact angle (<70°) for EVA (Examples 2, 3, 5), EnBA (Example 6), PE-Octene (Example 10), PP-E (Example 7), PP/B/E Example 9) but fail on PP-B Example 8). PP-B is not compatible with Unithox 450 due to poor miscibility of each other.

### Formulations with EVA

Table 4 shows the formulation using EVA with different VA content together with Unithox 450 and Escorez 5690. As seen, while different types of EVA may tune the viscosity range, they do not substantially impact the contact angles of the system, all below 70° for both fresh and aged conditions.

TABLE 4

| Example | 11 | 12 | 13 | 14 |
|---|---|---|---|---|
| Escorez 5690 | 45 | 45 | 45 | 45 |
| Unithox 450 | 5 | 5 | 5 | 5 |
| Ateva 1850A, 18% VA content | 49 | | | |
| Ateva 2842A, 28% VA content | | 49 | | |
| Ateva 3342, 33% VA content | | | 49 | |
| Ateva 4030, 40% VA content | | | | 49 |
| Evernox 10 | 1 | 1 | 1 | 1 |
| Viscosity @ 150° C. (cps) | 14,400 | 6725 | 6700 | 33,500 |
| Contact Angle - Fresh (°) | 29 | 37 | 37 | 42 |
| Contact Angle- 15 days at 60° C. | 31 | 34 | 47 | 47 |
| (°) | | | | |
| Comments | Pass | Pass | Pass | Pass but high |
| | | | | viscosity |

### Further Contact Angle Measurements

Table 5 shows the impact of the ethoxylate surfactant on the contact angle. Unithox 420, 450 and 480 all result in very low contact angle of hot melt adhesives.

TABLE 5

| Example | 5 | 15 | 16 |
|---|---|---|---|
| Escorez 5690 | 45 | 45 | 45 |
| Unithox 420 | | | 9 |
| Unithox 450 | 18 | | |
| Unithox 480 | | 18 | 9 |
| Ateva 2842A | 36 | 36 | 36 |
| Evernox 10 | 1 | 1 | 1 |
| Viscosity @ 150° C. (cps) | 2000 | 2500 | 2300 |
| Contact Angle - Fresh (°) | 31 | 34 | 24 |
| Contact Angle- 15 days at 60° C. (°) | 33 | 32 | 48 |
| Comments | Pass | Pass | Pass |

### Examples 17-19

Table 6 shows a formulation with plasticizers (examples 17-19). The addition of plasticizer provides better wetting properties. Ideally, the total content of the plasticizer is less than 20 wt % of the adhesive.

TABLE 6

| Example | 4 | 5 | 17 | 18 | 19 |
|---|---|---|---|---|---|
| Escorez 5690 | 45 | 45 | 40 | 35 | 30 |
| Unithox 450 | | 18 | 5 | 5 | 5 |
| Ateva 2842 | 54 | 36 | 49 | 49 | 49 |
| Calsol 5550 | | | 5 | 10 | 15 |
| Evernox 10 | 1 | 1 | 1 | 1 | 1 |
| Viscosity @ 150° C. (cps) | 10150 | 2000 | 4780 | 4280 | 3850 |
| Contact Angle (°) - Fresh | 91 | 31 | 30 | 31 | 31 |
| Contact Angle C. (°) - 15 days | 91 | 33 | 41 | 50 | 54 |
| at 60° | | | | | |
| Comments | Fail | Pass | Pass | Pass | Pass |

### Bond Strength

Table 7 shows the bond strength of laminate made by slot coating or spiral coating adhesives onto a general-purpose topsheet nonwoven then bonding to a core nonwoven. The coat weight is 8 grams per square meter, line speed 300 meter per min. The bond strength may be optimized by adjusting the content of polymer and surfactant.

TABLE 7

| Example | 5 | 12 |
|---|---|---|
| Bond strength by slot coated (gram/in) | 58 | 206 |
| Bond strength by spiral coated (gram/in) | 51 | 90 |

### Comparative Examples

Examples 20, 21, and 22 of Table 8 show the effect of conventional, low molecular weight, nonionic surfactant blend (ATMER 688 from Croda International) as described in WO 97/48779 and U.S. Pat. No. 6,380,292. The film contact angles of Examples 20, 21, and 22 are all greater than 70° at both fresh and after aging conditions. In contrast, Examples 9 and 13, made with an ethoxylate surfactant, Unithox 450, have a fresh contact angle below 70°.

TABLE 8

| Example | 9 | 13 | 20 | 21 | 22 |
|---|---|---|---|---|---|
| Escorez 5690 | 45 | 45 | 45 | 45 | 45 |
| Unithox 450 | 18 | 5 | | | |
| Atmer 688 | | | 5 | 5 | 18 |
| Vestoplast 508 | 36 | | | 49 | 36 |
| Ateva 3342, 33% VA content | | 49 | 49 | | |
| Evernox 10 | 1 | 1 | 1 | 1 | 1 |
| Viscosity @ 150° C. (cps) | 1950 | 6700 | 6600 | 5400 | 1160 |
| Contact Angle - Fresh (°) | 41 | 37 | 72 | 98 | 95 |
| Contact Angle- 15 days at 60° C. (°) | 35 | 47 | 80 | 98 | 97 |
| Comments | Pass | Pass | Fail | Fail | Fail |

What is claimed is:

1. An absorbent article for personal hygiene comprising a hydrophilic hot melt adhesive, wherein the hydrophilic hot melt adhesive comprises:
(a) an ethylene copolymer selected from the group consisting of ethylene vinyl acetate, ethylene n-butyl acrylate, ethylene octene, ethylene propylene, ethylene butene propylene, and mixtures thereof; and
(b) at least one nonionic, fatty alcohol ethoxylate surfactant having (i) a melting temperature of from about 70° C. to about 140° C., measured in accordance with ASTM D-127, (ii) a fully saturated linear C20 to C50 synthetic alcohol, (iii) an acid number below 100 mg KOH/g measured in accordance with ASTM E-222, and (iv) a molecular weight of about 400 to about 5000 Daltons.

2. The absorbent article for personal hygiene according to claim 1, wherein the hydrophilic hot melt adhesive comprises:
(a) from about 20% to about 90% by weight of the adhesive of the ethylene copolymer;
(b) from about 1% to about 25% by weight of the adhesive of the nonionic, fatty alcohol ethoxylate surfactant(s).

3. The absorbent article for personal hygiene of claim 1, wherein the hydrophilic hot melt adhesive comprises, by weight of the hot melt adhesive:
(a) from about 30% to about 70% of the ethylene copolymer; and
(b) up to about 20% of the nonionic, fatty alcohol ethoxylate surfactant(s).

4. The absorbent article for personal hygiene of claim 1, wherein the ethylene copolymer comprises at or consists of ethylene vinyl acetate.

5. The absorbent article for personal hygiene of claim 1, further comprising, by weight of the hot melt adhesive:
(a) from about 20% to about 70% of a tackifying agent,
(b) up to 20% of a plasticizer; and
(c) up to 3% of an antioxidant.

6. The absorbent article for personal hygiene of claim 5, wherein the plasticizer is selected from the group consisting of naphthenic oil, paraffinic oil, vegetable oil, animal oil, olefin oligomers, mixtures thereof and derivatives thereof.

7. The absorbent article for personal hygiene of claim 5, wherein the tackifying agent is selected from a hydrocarbon resin, a rosin ester resin, a terpene resin, and mixtures thereof.

8. The absorbent article for personal hygiene of claim 7, wherein the hydrocarbon resin is selected from the group consisting of C5 aliphatic resins, C9 aromatic resins, dicyclopentadiene resins, C5/C9 aliphatic/aromatic resins, and mixtures thereof.

9. The absorbent article for personal hygiene of claim 7, wherein the rosin ester resin is selected from rosin acids, rosin esters, hydrogenated rosin resins, dimerized rosin resins, modified rosin resins, and mixtures thereof.

10. The absorbent article for personal hygiene of claim 1, comprising in this order: a topsheet, optionally an acquisition layer, an absorbent core, and a backsheet; wherein the hydrophilic hot melt adhesive bonds at least two layers selected from:
the topsheet to the acquisition layer;
the acquisition layer to the absorbent core; and/or
the topsheet to the absorbent core.

11. The absorbent article for personal hygiene of claim 1, wherein the article comprises an absorbent core, the absorbent core comprising an absorbent material disposed in a core wrap, the core wrap having a top side and a bottom side, the top side being closer to the topsheet than the bottom side, and wherein the hydrophilic hot melt adhesive is disposed between the top side of the core wrap and the absorbent material layer and at least partially immobilizes the absorbent material to the top side of the core wrap.

12. The absorbent article for personal hygiene according to claim 1, wherein the article comprises a first nonwoven, and the first nonwoven is attached to another component of the article by the hydrophilic hot melt adhesive.

13. The absorbent article for personal hygiene according to claim 12, wherein the first nonwoven is attached by the hydrophilic hot melt adhesive to one selected from a second nonwoven, cellulose fibers, or superabsorbent particles, and combinations thereof.

14. The absorbent article for personal hygiene according to claim 1, wherein the hydrophilic hot melt adhesive exhibits a Fresh contact angle and an Aged film contact angle with distilled water of less than 70 degrees, as measured by the Contact Angle Test.

15. A package comprising a plurality of absorbent articles for personal hygiene according to claim 1.
